(19) United States
(12) Patent Application Publication
(10) Pub. No.: US 2007/0280418 A1
(43) Pub. Date: Dec. 6, 2007

(54) Coupled Carriers for Enhancing Therapy

(75) Inventor: Michael D. Weil, Fort Collins, CO (US)

Correspondence Address: MICHAEL WEIL, 584 W. DOUGLAS ROAD, FORT COLLINS, CO, 80524, US

(73) Assignee: Sirius Medicine, LLC, Fort Collins, CO (US)

(21) Appl. No.: 11/758,001

(22) Filed: Jun. 5, 2007

Related U.S. Application Data
(60) Provisional application No. 60/803,974, filed on Jun. 5, 2006.

(51) Int. Cl. A61N 5/10; A61K 51/00; A61M 36/14

(52) U.S. Cl. 378/65; 424/1.11

(57) ABSTRACT

Radiation has been used for many years to treat cancer patients. In the case of ionizing electromagnetic radiation, the incidental, extra dose to tissues surrounding the tumor is significant. The aim of this invention is to provide compounds and methods to enhance the absorbed radiation dose ratio, or other therapeutic compounds, in tumors verses normal tissue. The system concentrates contrast agents with high atomic number elements preferentially at the site of the tumor prior to administering radiotherapy, or preferentially concentrates other therapeutic compounds in the abnormal region. The agents are concentrated in a pathologic lesion following systemic or direct administration. Interaction of the ionizing radiation with the coupled compounds of this invention results in a significantly higher radiation dose to the tumor compared to surrounding tissues. The result is greater therapeutic efficacy with fewer side effects following treatment with low-energy radiation, or other agents. These compounds permit diagnostic uses in combination with the therapeutic use.

### RELATED U.S. APPLICATION DATA

This application claims priority of provisional application No. 60/803,974 filed Jun. 5, 2006 and entitled, Coupled Carriers for Enhancing Therapy.

### REFERENCES CITED

### U.S. Patent Documents

| | | |
|---|---|---|
| 5,008,907 | April 1991 | Norman et al. |
| 5,521,289 | May 1996 | Hainfeld et al. |
| 5,911,969 | May 1999 | Axworthy et al. |
| 5,914,312 | May 1999 | Axworthy et al. |
| 5,919,135 | July 1999 | Lemelson |
| 5,976,535 | November 1999 | Fritzberg et al. |
| 6,125,295 | September 2000 | Cash and Weil |
| 6,207,133 | March 2001 | Reszka et al. |
| 6,369,206 | April 2002 | Leone and Hainfeld |
| 6,366,801 | April 2002 | Cash and Weil |
| 6,416,738 | July 2002 | Theodore et al. |
| 6,645,464 | November 2003 | Hainfeld |
| 6,955,639 | October 2005 | Hainfeld and Slatkin |
| 7,078,013 | July 2006 | Axworthy et al. |
| SN 11/671,222 | February 2007 | Weil et al. |

### OTHER PUBLICATIONS

Iwamoto et al., “Radiation dose enhancement therapy with iodine in rabbit VX-2 brain tumors,” Radiother. Oncol. 8:161, 1987, Elsevier.

Santos Mello et al., “Radiation dose enhancement in tumors with iodine,” Medical Physics 10:75, 1983, Am. Assoc. Phys. Med.

Hnatowich et al., “Investigations of Avidin and Biotin for Imaging Applications, ” J. Nuc. Med. 28:1294, 1987, Soc. Nuc. Med.

Solberg et al., “Calculation of radiation dose enhancement factors for dose enhancement therapy of brain tumours,” Phys. Med. Biol. 37:439, 1992, Inst. Phys. Pub.

Rose et al., “First radiotherapy of human metastatic brain tumors delivered by a computerized tomography scanneer (CTRx),” Int. J. Radiat. Oncol. Biol. Phys. 45:1127, 1999, Elsevier.

Zhang et al., “Pretarget radiotherapy with an anti-CD25 antibody-streptavidin fusion protein was effective in therapy of leukemia/lymphoma xenografts,” Proc. Natl. Acad. Sci. USA 100(4):1891, 2003, Natl. Acad. Sci. USA

Hainfeld et al., “Gold nanoparticles: a new X-ray contrast agent,” Brit. J. Radiol. 79:248, 2006, Brit. Inst. Radiol.

Weil et al., “Contrast-Enhanced Radiotherapy with GMCSF for Advanced Human Cancer,” In Preparation, 2007.

### FIELD OF THE INVENTION

The present invention relates to the field of medical ablation of lesions that are detrimental to the body. More specifically this invention relates to the use of linked agents, such as nanoparticles, liposomes or radiosensitizers, with affinity for each other to first specifically localize within lesions, and then to attract, or enhance, therapeutic concentrations of other linked compounds relative to the surrounding normal tissues. The following set of delivered compounds is cytotoxic by itself, or becomes cytotoxic upon interaction with radiation.

### BACKGROUND OF THE INVENTION

Radiation is a standard treatment for localized cancer. Radiotherapy exposes a diseased part of the body to ionizing radiation. The goal is to destroy abnormal cell, although healthy cells frequently are exposed to high radiation doses as well. This can produce significant toxicity. In order to increase the ratio of the dose to the lesion versus normal tissue, radiation is often delivered to the tumor from multiple angles. This reduces injury to overlying tissue. However the x-rays are spread around the tumor and overshoot the target. Ideally, healthy cells are better able to repair this lower-level damage and remain viable while diseased cells die. Nevertheless, there is significant elevation in overall dose to normal tissues (i.e., the integral dose).

Radiotherapy is most often performed with high energies. The interactions of high-energy radiation are relatively independent of the composition of the body. This is because radiation above 100 keV interacts with tissues via Compton scattering, which is modestly affected by tissue—a photon transfers part of its energy to an electron, the photon is deflected and continues with less energy. The electron and th lower energy photon continue to produce more secondary ionizations.

Low-energy x-rays (≦100 keV) are composition sensitive because the dominant interaction is the photoelectric effect. In this case, a photon transfers its energy to an electron and the photon disappears. The resulting photoelectron and characteristic x-rays produce secondary ionizations. The many secondary ions produced are important in producing biological effects. Elements with high atomic number (Z) will preferentially absorb x-rays of this energy relative to the lower Z elements that make up soft tissues.

The use of contrast agents to enhance the photoelectric effect of low-energy x-rays for tumor treatment has been reported (U.S. Pat. Nos. 6,125,295 and 6,366,801; Santos Mello et al., “Radiation dose enhancement in tumors with iodine,” Medical Physics 10:75, 1983, Am. Assoc. Phys. Med.; Iwamoto et al., “Radiation dose enhancement therapy with iodine in rabbit VX-2 brain tumors,” Radiother. Oncol. 8:161, 1987, Elsevier, Solberg et al., “Calculation of radiation dose enhancement factors for dose enhancement therapy of brain tumours,” Phys. Med. Biol. 37:439, 1992, Inst. Phys. Pub.; Rose et al., “First radiotherapy of human metastatic brain tumors delivered by a computerized tomography scanner (CTRx),” Int. J. Radiat. Oncol. Biol. Phys. 45:1127, 1999, Elsevier). The basic technique is to deliver a contrast agent to a tumor mass prior to delivering the radiation dose. The contrast agent has as one of its components an element with a high atomic number (Z), such as iodine, tungsten, gold or bismuth. The interaction between the ionizing radiation and the greater cross-section of the high Z material creates additional ionizations that result in higher radiation dose absorption, and therefore, cell killing at the tumor site. Contrast agents proposed for such an application include, iodinated x-ray contrast agents, gadolinium (Gd)-based imaging agents, and tungsten, gold or bismuth particles.

Following the interactions between low-energy x-rays (keV) with a high Z material, the resulting dose enhancing ionizations take place over a very small distance. This distance (d) can be calculated as follows:

$$d = \frac{( 0.046 ) {( keV )}^{1.75}}{{( δ )}_{tissue}}$$

When the x-ray energy (keV) is in the diagnostic range=35-60 keV, and (δ)ₜᵢₛₛᵤₑ=1.3 keV/μm, for tissue-equivalent matter, then d=18-46 Mm. Since a cell is ˜8 μm diameter, the photoelectrons produced by irradiating a high Z element in this invention travel a distance of ˜5 cells (=46 μm/8 μm). As a result, high dose radiation is delivered very tightly about the contrast agent.

Tumors have been loaded with sufficient contrast by direct, intralesional injection. Needles are localized in tumors with image guidance, such as a CT scanner. The contrast is then injected to distribute the high Z material in a reasonable concentration to enhance a low-energy radiation dose to lethal levels. Contrast-enhanced radiotherapy (CERT) performed with intratumoral injection has been reduced to practice (Weil et al, In Preparation, 2007).

However, while intratumoral contrast injection is useful, it has drawbacks. It requires a skilled practitioner to place needles in tumors and a avoid critical normal structures. The varying composition of cancerous masses can result in spotty filling of the mass with direct injection. The high intratumoral hydrostatic pressure can result in backflow during direct injections and result in superficial extravasation of contrast. Furthermore, since contrast can exit the tumor quickly, radiation needs to be delivered soon after contrast injection is completed.

To avoid the shortcomings of intratumoral injection, this invention devises contrast media capable of achieving intratumoral concentration that is sufficient for CERT with intravenous infusion. This application will be more fully described because it requires delivery of the highest amounts of a compound to the target for clinical efficacy, and therefore, represents the most stringent test for the invention. Of note, the capability of this invention to deliver sufficient concentration of a high Z atom to a tumor for CERT, also includes its use for delivery to other lesions (e.g., pathologic plaque in the blood vessels or nervous system) of other therapeutic agents, such as chemotherapy, immunotherapy, proteins, peptides, toxins, nucleic acids, radiosensitizers and radioisotopes with higher specificity and better outcome than possible at present.

Conventional medical contrast agents have no specific affinity for cancerous masses. When delivered systemically by intravenous infusion, contrast media passively diffuses into malignant lesions through disorganized and leaky tumor vasculature. However, tumors have elevated hydrostatic pressure because their cells are tightly packed. Any contrast agent delivered to a tumor by way of the blood stream must overcome these significant obstacles to flow. Moreover, the residence time of conventional agents in a tumor is short. Therefore, the concentration of a high Z material in a lesion after intravenous infusion is normally limited to <1% weight per volume of the tumor mass. This level of tumor uptake with conventional contrast agents is adequate for diagnostic imaging, but is inadequate for useful enhancement of radiotherapy.

There have been efforts to develop better tumor uptake of high Z materials and radionuclides for imaging (Hnatowich et al., “Investigations of Avidin and Biotin for Imaging Applications,” J. Nuc. Med. 28:1294, 1987, Soc. Nuc. Med.) and therapy (Zhang et al., “Pretarget radiotherapy with an anti-CD25 antibody-streptavidin fusion protein was effective in therapy of leukemia/lymphoma xenografts,” Proc. Natl. Acad. Sci. USA 100(4):1891, 2003, Natl. Acad. Sci. USA) using the intravenous route. Most of these compounds are designed to have improved specificity for cancer cells. They typically use a biological carrier such as a protein, or a monoclonal antibody, antibody fragments, peptides and nucleic acid-based aptamers, which bind with specific affinity for a component of the tumor or its vasculature. For example, if a tumor has a distinct surface target for an antibody, it is possible to attach a payload of iodine, other heavy element or radionuclide, for better delivery to a lesion. Alternatively, “pretargeting” approaches separate the radionuclide from the antibody by first infusing an unlabeled antibody followed by a radiolabeled molecule, which binds with high affinity to the antibody or a modified component of it. These “pretarget” approaches have had modest success imaging a few cancer types. However, the efficiency of localization and uptake is too low to enhance radiotherapy unless the heavy elements are radioactive (U.S. Pat. Nos. 5,911,969, 5,914,312, 5,976,535, 6,416,738 and 7,078,013). On the other hand, radioactive isotopes have the significant disadvantage of delivering high radiation doses to the liver and kidneys when the compounds are cleared from the body (comparable problems can result when other cytotoxic agents, e.g., chemotherapy or toxins, are attached to biological carriers).

Finding unique markers for cancer cells is difficult. A more straight forward approach to improve tumor uptake of intravenous contrast is to increase the payload of contrast that can passively diffuse into a cancer. High Z materials have been encapsulated, e.g., liposomes, or packed together as nanoparticles, which are 0.5-400 nm in diameter (U.S. Pat. Nos. 5,521,289 and 6,369,206). Adequate payloads of contrast can be delivered with nanoparticles for imaging and the technique has been proposed as a method for enhancing radiotherapy (U.S. Pat. Nos. 6,645,464 and 6,955,639).

However, intravenous delivery of high Z materials with gold nanoparticles (even when attached to a targeting molecule like an antibody) achieve no more than an order of magnitude less than the concentration required for clinically useful contrast-enhanced radiotherapy. For example, Hainfeld et al (“Gold nanoparticles: a new x-ray contrast agent,” Brit. J. Radiol. 79:248, 2006, Brit. Inst. Radiol.) employed intravenous delivery of gold nanoparticles (1.9 nm diameter) into experimental mouse tumors. The experiments injected 54 mg of gold through a vein in the animals' tails. After 15 minutes, gold concentration in the tumor was 4.2% of the injected dose per gram of measured tissue. This is equal to 2.27 mg/gm, which expressed as grams of gold per 100 grams of tumor,=0.23% weight/volume [Note: the concentration of the heavy element is better expressed as the “fraction by weight” in the compound to calculate the radiation dose enhancement. The fraction by weight of an atom, or molecule, is derived from the concentration of the heavy element divided by the density of the compound. However, concentration expressed as “percentage of weight per volume” is the standard medical convention. Therefore, concentrations herein are described as grams per 100 ml of solution, or in percent]. This concentration of gold in a tumor is approximately a factor of 10 less than the minimum threshold which is clinically useful for CERT.

At this point in time, targeting agents are not optimal for the CERT application. Thus, there is a need for better delivery and concentration of heavy atoms in pathologic lesions to enhance the absorbed radiation dose for cancer treatment. The same technology can readily be applied to improve delivery of other compounds, such as chemotherapy, immunotherapy, proteins, peptides, toxins, nucleic acids, and radioisotopes to tumors and other lesions.

### SUMMARY OF THE INVENTION

The aim of this invention is to specifically deliver therapeutic amounts of agents to pathologic lesions via interactions that are independent of the histology, or traits of the lesion. Furthermore, therapeutic compounds and agents, such as a high Z metal or cytotoxic compound, will be made capable of achieving a minimal required threshold concentration and distribution throughout a lesion following intratumoral or intravascular delivery, including intravenous, intra-arterial or other systemic routes. this will be achieved by using and accumulating coupled, carrier complexes with adequate payloads of the therapeutic agent in the tumor.

Achieving sufficient concentration of the carriers in the target will be done in steps. The first carrier will passively diffuse into a tumor and normal tissue (FIG. 1). Following an interval to allow the higher blood flow in the normal tissue to wash out the carrier from the normal tissue (FIG. 2), a second carrier will be infused. The second carrier will be modified with molecules on its surface that recognize and bind complementary molecules on the first carrier's surface (FIG. 3). The interaction of binding molecules between the first and second carriers will be specific and have high affinity. As such, the first carrier will passively diffuse into a tumor without having any specific affinity for the tumor. The intratumoral first carrier will then be used as the target for the second carrier. The second carrier will target the known surface molecules on the first carrier. Specific antigens of the tumor are not needed, or used, to accumulate the second carrier in the tumor. And, as the second carrier specifically locates the first carrier in the tumor, the payload of the carried therapeutic agent is increased in the tumor target (FIG. 4). These steps are repeated (FIG. 5), and can include third or more carriers, to produce a minimal threshold concentration of carried therapeutic agent in the target lesion (FIG. 6).

In another embodiment of this invention, the first carrier could employ specific biologic molecules, such as a protein, or a monoclonal antibody, antibody fragments, peptides and nucleic acid-based aptamers, which bind with specific affinity for a component of the tumor or its vasculature to localize the first carrier to the lesion. In this case, the second carrier will still be modified with molecules on its surface that recognize and bind complementary molecules on the first carrier's surface (FIG. 3). The interaction of binding molecules between the first and second carriers will be specific and have high affinity. Here, the first carrier selectively targets a lesion by having specific affinity for the tumor. The intratumoral first carrier will then be used as the target for the second carrier. The second carrier will target the known surface molecules on the first carrier. Once again, specific antigens of the tumor are not needed, or used, to accumulate the second carrier in the tumor. And, as the second carrier specifically locates the first carrier in the tumor, the payload of the carried therapeutic agent is increased in the tumor target. These steps are repeated, and can include third or more carriers, to produce a minimal threshold concentration of carried therapeutic agent in the target lesion.

The mechanisms for coupled complexes for the carriers of this invention are divided up into several categories:

a) Carriers with specific affinity between paired ligand-receptor molecules on their surfaces, e.g., linked “male-female” molecules. The carrier complexes of this invention are particles of heavy element, such as nanoparticles or liposomes, biological and non-biological macromolecules, which can be coupled or linked via attached targeting molecules, e.g., antibodies, antibody fragments, protein-substrate complexes, biotin-binding proteins, biotin-streptavidin complexes, peptides, ligands, receptors, aptamers, nucleic acid-based aptamers, complementary nucleic acids, chelates or small molecules.

b) Carriers capable of targeting entire first carriers. In this case, the second carrier has a structure or attached molecule that recognizes the first carrier itself. The carrier complexes of this invention are particles of heavy element, such as nannoparticles or liposomes, biological and non-biological macromolecules, which can be coupled, or linked via specific structure, or attached targeting molecules, E.G., antibodies, antibody fragments, protein-substrate complexes, biotin-binding proteins, biotin-streptavidin complexes, peptides, ligands, receptors, aptamers, nucleic acid-based aptamers, complementary nucleic acids, chelates or small molecules.

c) Magnetic particles. The carriers can be magnetized, or made of metals capable of being attracted to magnetized particles. The second carrier localizes to the first carrier in a lesion by magnetic attraction.

The targeting carrier complexes of this invention have no special affinity for cancer cells, but they can carry a high payload of heavy elements, or cytotoxic compounds, to the site of the cancer, and/or have the ability to penetrate inside the cell. In one application, the invention is used to deliver agents that are themselves cytotoxic, such as chemotherapeutic compounds, immunotherapy, or radioisotopes, to the lesion. In another application, ammounts of non-toxic, radiation-enhancing contrast agent (high Z material) sufficient for CERT are delivered to lesions. These contrast agents then produce toxicity by subsequent interaction with external beam, or radioisotope radiation.

The delivery with this invention of sufficient contrast agent for CERT is used as the primary example because it requires the highest concentration in the target for efficacy. If the invention is adequate for this application, it is readily applicable to the delivery of other agents, e.g., cytotoxic compounds, with lower thresholds of efficacy.

Contrast agents and tumor targeting techniques at present do not achieve adequate tumor concentration of heavy atoms for CERT if given intravenously. In the example from Hainfeld et al (“Gold nanoparticles: a new X-ray contrast agent,” Brit. J. Radiol. 79:248, 2006, Brit. Inst. Radiol.) employing intravenous delivery of gold nanoparticles (1.9 nm diameter)into experimental mouse tumors; they measured 4.2% of the intravenously injected gold dose per gram of measured tumor. Since the injected dose was 54 mg, this was equal to 2.27 mg/gm in the tumor, and 0.23% weight/volume. However, for practical implementation of CERT, it is necessary to obtain ˜2.5-30% weight/volume of a heavy element in a tumor. Therefore, if CERT was attempted for a tumor, which had been filled with gold particles as reported by Hainfeld et al, the dose enhancement would be 10-100 times less than required for clinical efficacy (Weil et al, In Preparation, 2007).

To provide sufficient radiation-enhancing contrast agent at the site of a tumor for CERT, it is necessary with intravenous delivery to increase its concentration 10-100-fold. The compounds and methods of this invention are designed to reach the requisite 1-2 orders of magnitude increase in intratumoral heavy atom concentration and resulting radiation dose enhancement.

One approach to improving uptake of high Z agents by a tumor in this invention involves taking advantage of the fact that nanoparticles remain in tumors significantly longer than in normal tissues. For example, Hainfeld et al showed that over 24 hours the tumor retained 64% of peak value of gold from the injected dose. Therefore, in the mouse experiment, it would be expected that 24 hours after intravenous injection the tumor retained gold=0.15% weight per volume (=0.23% weight/volume initially ×0.64 at 24 hours). As noted above, this value is too low (by at least an order of magnitude) to use for CERT. However, it can be turned into a significant starting value to use with CERT, if the initially deposited particle is subsequently targeted by a second agent with a complementary binding affinity for the first, and also carries an additional payload of high Z elements (the high Z material can be the same or different in the two particles). The invention includes methods and calculations to optimize the radiation deposition through choice of the appropriate high-Z element.

A distinguishing advantage of the described two-component system is that the interactions used to deliver th high-Z material (or other therapeutic molecule) to the tumor are chosen by the user and are not dictated by the targeted lesion. The first component is delivered by passive diffusion and is retained non-specifically in the lesion (though specific targeting can be used for the first carrier as well). The second component is then delivered via its affinity for the first. On the other hand, current approaches require an antibody or other delivery molecule with affinity for a constituent of the tumor or tumor vasculature that distinguishes the target from normal tissues. In this conventional case, the choice of delivery molecule is dictated by the availability of such differentiating constituents. For example, some tumor-associated antigens have been targeted by antibodies. However, different tumor types have different antigens requiring different antibodies, and many have no antigens identified to date. Conversely, this invention imposes the differentiating constituent on the tumor, independent of tumor type. This allows the user to choose an interaction tht has both high specificity and affinity, and is independent of tumor histology.

### BRIEF DESCRIPTION OF THE FIGURES

FIG. 1. Carrier 1 with target 1 distributes in the tumor and normal tissue upon systemic administration.

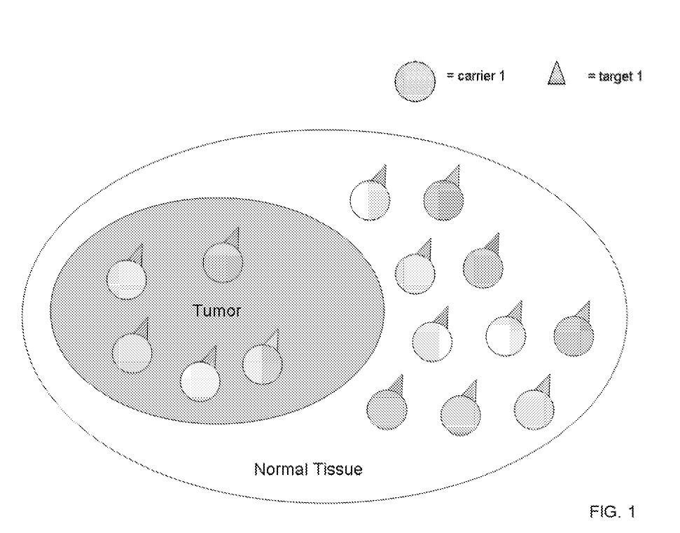

FIG. 2. Carrier 1 with target 1 remains in the tumor after that in the normal tissues has cleared.

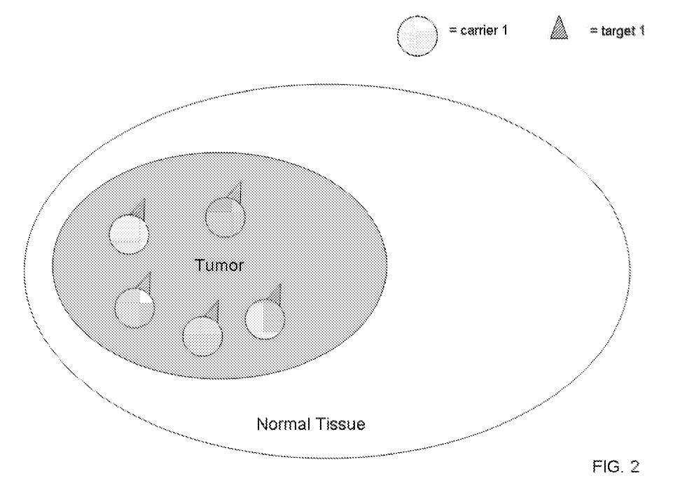

FIG. 3. Carrier 2 with specific binding entity for target 1 distributes in the tumor and normal tissue upon systemic administration.

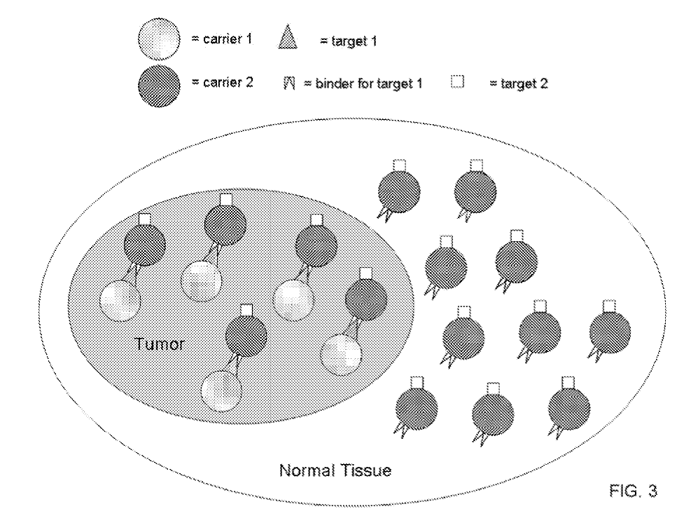

FIG. 4. Carrier 2 with specific binding entity for target 1 remains in the tumor due to its affinity for target 1 after that in the normal tissues has cleared.

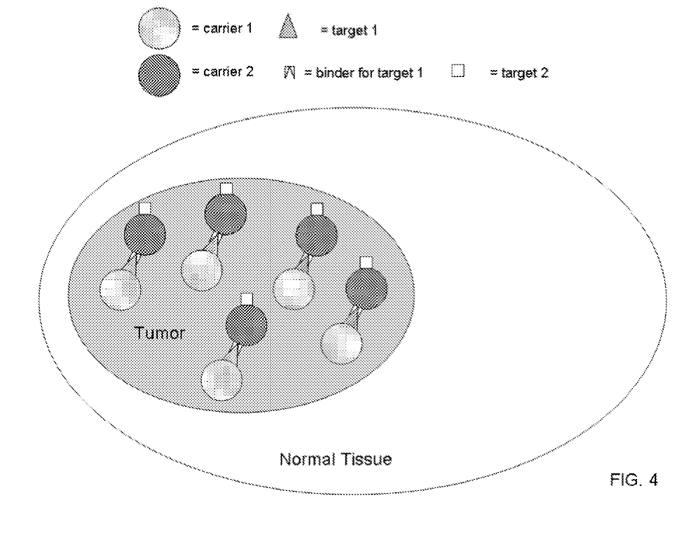

FIG. 5. Carrier 3 with specific binding entity for target 2 distributes in the tumor and normal tissue upon systemic administration.

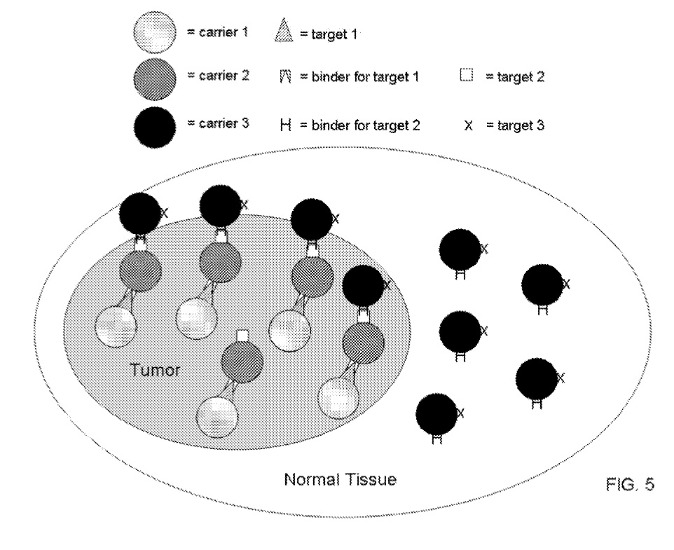

FIG. 6. Carrier 3 with specific binding entity for target 2 remains in the tumor due to its affinity for target 2 after that in the normal tissues has cleared.

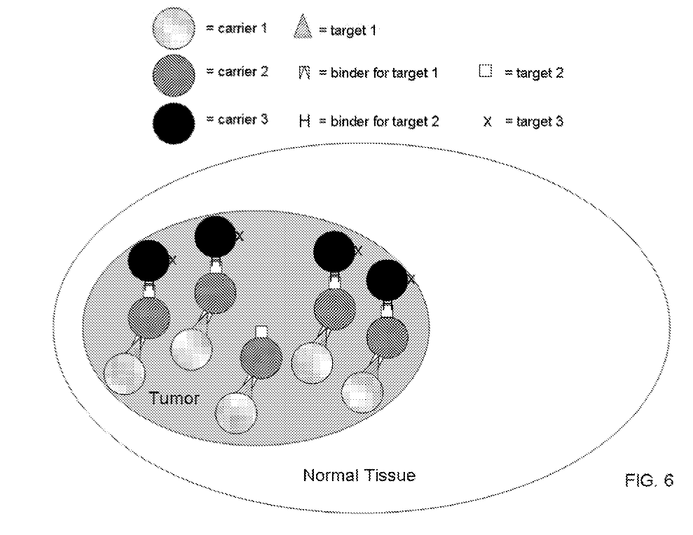

### DETAILED DESCRIPTION OF THE INVENTION

Binary, or coupled, targeting agent of this invention consist of two sets of nanoparticles, or liposome or other biological (e.g., viral particles) or non-biological macromolecules (e.eg., silicon particles): each set is made of a high Z element, and each set of nanoparticle, in one instance, is complexed to different complementary molecules, e.g., “lock and key” molecules, which are capable of binding to its target on the other nanoparticles with high, specific affinity. Preferably, the pharmaceutical compounds comprise a heavy element, or a rare earth heavy element, or other therapeutic agent, in a nanoparticle, liposome or other macromolecular compound, complexed with an antibody, antibody fragment, protein, peptide, biotin-binding protein, streptavidin, ligand, receptor, nucleic acid-based aptamer, complementary nucleic acids, chelate, small molecule, or other complementary specific molecular binding units.

The nanoparticles, in one embodiment fo this invention, can be made of high Z elements such as gold or tungsten. Other preferred heavy elemental ions include atoms chosen from the lanthnide series, or other high Z material. For example, La, Ce, Pr, Pm, Nd, Sm, Eu, Gd, Tb, Dy, Ho, Er, Tm, Yb, Lu and Ag, Ir, Pt, Au or Bi are heavy atoms of this invention. Other heavy elemental ions can also be used, if they have high atomic numbers and are not toxic when complexed. Non-liminting examples for this invention are Y, In, Re, Sn, La, Ac, Pd and Ag.

In this embodiment of the invention, the first agent is taken up by the tumor through passive diffusion or with a tumor targeting molecule, such as a protein, peptide, ligand, receptor, chelate, folate, a nucleic acid-based aptamer, complementary nucleic acids, antibody, or antibody fragments on the surface. In addition, the first agent has on its surface a molecule that can act as a receptor, or specific binding site, or ligand, e.g., biotin, for a targeting molecule on the second agent, which also carries a payload of high Z atoms. The targeting molecule on the second agent is specific for the first agent and is designed to bind to the target on the surface of the first agent localized in the tumor. The second agent is not designed to target the tumor per se. The targeting molecule on the surface of the secondary nanoparticles can include: proteins, peptides, ligands, receptors, streptavidin, biotin-binding proteins, nucleic acid-based aptamers, complementary nucleic acids, antibodies, antibody fragments, chelates and small molecules. The second agent can be made of the same high Z atoms, or different high Z atoms, a chemotherapeutic agent, immunotherapeutic agent, peptides, a radioisotope, or an aggregate of agents (e.g., in a liposome). In general, the second agent will carry a greater payload of heavy Z atoms. In this way, as the second agent finds and specifically binds the first agent in the tumor, a significantly greater amount of high Z atoms will be concentrated in the tumor target.

In the example of gold nanoparticles cited above, the addition of 20 gold nanoparticles to a secondary agent would enhance the gold levels at the tumor following the infusion of the first agent (individual gold nanoparticles in this case) 20-fold: from 0.15% to 3% weight/volume. This is a threshold concentration to perform CERT in a practical manner. Since as many as 250 nanoparticles can be attached to an antibody, it could be possible to achieve intratumoral gold concentrations>30% weight/volume. The use of a spreading agent could improve the distribution of large collections of nanoparticles on second agents. We have employed CERT with spreading agents, such as hyaluronidase (Vitrase™, Ista Pharmaceuticals, Irvine, Calif.), to facilitate the distribution of contrast in tumors (Weil et al, In Preparation, 2007).

It may be difficult for very large collections of nanoparticles to diffuse into a tumor. A 20 nm diameter gold nanoparticle is approximately the size of an immunoglobulin molecule, which is the size of a large molecule that might readily diffuse into a solid tumor. However, as more nanoparticles of a given diameter are added to a second agent it could become too bulky to pass into the tumor and bind to the first agent. Therefore, another component of this invention is to include a binding target on the second agent. In this way, second agents will be able to target the intratumoral first agent and also, become the target of another second agent, or a third agent. If the second agents can bind to one another, they will have to be delivered in low concentration, so as not to clump together. A third agent targeting the second agent would not have that problem. It would likely be able to carry the highest payload, since it would only need to find second agents and presumably not have to penetrate as deeply in the tumor. Alternatively, a series of second agents could be infused at intervals with increasing nanoparticle payload. Smaller loads would be infused first, and progressively greater payloads would follow in sequence.

The method of the present invention includes the step of administering to a recipient with a solid lesion, an initial dose of primary nanoparticles, a tumor-seeking agent, which is not a radiopharmaceutical (a radiopharmaceutical is defined herein to mean a complex of a radionuclide and a ligand, which targets bone of soft tissue), i.e., neither the agent nor the heavy elements are radioactive. The tumor could be primary, or metastatic. After allowing the localization of the primary agent to the lesion and the loss of the primary nanoparticles from the normal tissue, the secondary, nanoparticles-seeking agent, is delivered systemically. If needed a third agent targeting the second agent would be used.

The infused lesion is imaged and the concentration of high Z material in the target is determined. If the imaging is done with a CT scanner the CT numbers (Hounsfield Units) can be used to calculate the dose enhancement factor for CERT. Likewise, if a different type of digital detector is employed the dose enhancement factor can be derived from the attenuation coefficients measured with multiple beams. If, after calculating the dose enhancement factor for CERT with this method, the potential enhancement is too low, then additional infusions of secondary, or third nanoparticle-seeking agents can be delivered. The process is repeated until there is sufficient high Z material in the target to produce adequate dose enhancement. Following the step-wise delivery of secondary agents and measurement of uptake until a minimal concetration threshold is reached; the area of the lesion is treated with radiation. The radiation can be delivered by either an external radiation beam, e.g., an x-ray beam, or via brachytherapy (localized, radioactive seeds). This invention does not employ this invention are defined herein to mean a complex of a heavy element and a ligand which targets complementary “receptors” located on other complexes of heavy element deposited a priori in tumor tissue.

Administration of coupled nanoparticles as described in this invention is accomplished by intravenous (i.v.), interstitial, or intramuscular administration. In order to increase the amount of heavy atom concentration at the site of the tumor, the coupled agents of this invention can be administered at multiple time points, or by a slow continuous infusion in the bloodstream keeping a high concentration in the bloodstream until the desired amount of contrast agent is in the tumor. Alternatively, a tourniquet can be used to isolate the site of the tumor prior to administering the dose to increase uptake in the tumor. The measurement of the needed concentration of the agent at the target can be determined by imaging with a CT scanner, or other imaging device as noted above.

The types of tumors that can be treated by this invention include primary and metastatic bone and soft tissue tumors. When the location of these tumors is known, one modality of treatment is to administer the coupled agent, then concentrate the radiation to the area of the tumor, thus increasing the ratio of absorbed radiation dose in the target versus normal tissue. In other cases, where many tumors are in need of treatment, or where there is disseminated disease, it is possible to administer the coupled agents then give relatively low radiation to the whole body. This way of treating the patient may treat micro-metastatic sites, or small tumors, before they grow into bigger and less treatable tumors.

Targeting soft tissue cancers has been done by using biological targeting moieties. This is accomplished by the use of a targeting moiety such as a protein, or a monoclonal antibody, or fragments thereof. Even though this technology can be used to deliver high Z materials tumor cells, the amount that can be delivered to the tumor cell is relatively low, and is not enough to cause a significant enhancement of dose when delivering radiation. In addition, biological targeting moieties can be complex to produce, are fragile, and may elicit an allergic response from the body. There is a need for new contrast agents for the purpose of enhancing the radiation dose absorbed by the target. It is an additional aim of this invention to provide simple low molecular weight contrast agents that can deliver high Z elements directly to cancers.

Another critical component of this invention is quantification and dosimetry of the delivered dose of radiation. These parameters are influenced by the residence time of the targeting agent in the tumor and are dependent upon the kinetics of diffusion out of the target site. Other benefits of the invention include both extracellular and possible intracellular distribution of the agents (conventional contrast goes no further than the extracellular space). Use of coupled carrier complexes in this manner can enhance the effect of external beam radiation or brachytherapy after direct injection into the tumor, IV injection and topical applications. These kinds of advantageous attributes are not possible with known iodinated x-ray contrast agents or gadolinium-based MR contrast agents.

It is a further aim of this invention to provide targeting agents that have can deliver a high payload to the site of the tumor. This is achieved by a variety of technologies. Liposomes, gold particles (see also U.S. Pat. Nos. 6,125,295; 6,366,801 and 6,955,639), viral particles, and silicon particles are examples of carriers for high payloads of both targeting moieties and either imaging or therapeutic agents. In many cases for the above mentioned technologies, the strategy is to attach targeting moieties plus radioactive atoms to the macromolecule and administer the formulation into patients with cancer. Even though diagnostic applications for these systems are viable, therapeutic uses have been hampered by the high accumulation of these agents in the liver and other parts of the reticuloendothelial system. The high doses to these organs when using radioactive targeting moieties limit the use of the technology in this way. The utility of the coupled high payload systems, especially absent attached radioactive isotopes, for enhancing the effect of radiation therapy has not been taught elsewhere.

The use of high payload systems with the ability to target cancer is another aim of this invention. High Z materials, or cytotoxic agents, can be loaded in the carriers. Many payload systems and coupled carrier combinations are possible. This invention is not limited to the carrier system or the method by which it is derivatized. The aim is to administer a targeted, high payload system containing a large number of targeting molecules and high Z atoms to allow adequate uptake by the tumor. After an ample time for uptake, a patient is given a dose of localized, therapeutic radiation.

Treatment of brain cancers (primary and metastatic) is a potential application for CERT as minimizing damage to healthy brain tissue would be very beneficialo. The non-radioactive form of IOTREX™, sodium 3-(125I)iodo-4-hydroxybenzenesulfonate (the iodinated compound used with GliaSite™) in the treatment of brain cancer could be used in this method. Although, the targeting ability of the carriers might not be as direct as in lesions in the rest of the body, it could help substantially to minimize damage to healthy brain tissue. This has been demonstrated by treating a patient with brain tumors during a Phase I study in 1999 (Weil et al, In Preparation, 2007).

The radiation dosimetry can be optimized by choice of high Z atom in the chelate. The dose enhancement factor (DEF) of the high Z element (Z) is determined by the equation:

$$DEF = \frac{{( {μ}_{en} / ρ )}_{z} ⋆ ( {f}_{z} ) + {( {μ}_{en} / ρ )}_{target} ⋆ ( 1 - {f}_{z } )}{{( {μ}_{en} / ρ )}_{tissue}}$$

Where (μₑₙ/ρ)z, (μₑₙ/ρ)_{target} and (μₑₙ/ρ)ₜᵢₛₛᵤₑ are the mass energy-absorption coefficients (MEAC) of the high Z element of choice, the target and tissue, respectively at the employed beam energy; and f_{Z} is the fraction by weight of the high Z element. Since the x-rays from a medical source are a spectrum of energies, the MEAC values are calculated over that spectrum to accurately determine the DEF.

The penetration of the radiation through tissue will decrease the flux and also change the spectrum by hardening the beam, i.e., the average beam energy increases a lower energy photons are attenuated and higher energy photons relatively predominate. As a result of the radiation's path, the beam spectrum changes with tissue type and depth, as well as high Z atom type, concentration and volume in the target. In clinical practice, these variables, are accounted for and the DEF is calculated with planning software.

What is claimed is:

1. A method for increasing the effectiveness of medical therapy, comprising the steps of:
a. delivering an effective amount of a targeted first agent to a diseased tissue; and then
b. delivering an effective amount of a complementary second agent, which specifically targets and links to the first agent within the diseased tissue; and then
c. to attract, or enhance, therapeutic concentrations of other linked compounds in the lesion relative to the surrounding normal tissues.

2. The method according to claim1, wherein the targeted first or targeting second agent is a nanoparticle, a liposome, a high payload targeting system, a carrier, a coupled carrier, other coupled carrier complexes, or a combination thereof.

3. The method according to claim 1, wherein the first agent passively diffuses into a lesion.

4. The method according to claim 1, wherein the first agent specifically targets a tumor.

5. The method according to claim 4, wherein the first agent's tumor-specific localizing molecule can comprise:
a. a protein, peptide, ligand, receptor, chelate, folate, small molecule, nucleic acid-based aptamer, commplementary nucleic acid, antibody, or antibody fragment on the surface.

6. The method according to claim 1, wherein the targeted first agent has a molecule on its surface to act as a receptor, or specific binding site, or ligand, or antigen for a complementary targeting molecule on the second agent.

7. The method according to claim 6, wherein the complex of the first agent's receptor with a complementary molecule on the second agent can comprise:
a. antibodies, antibody fragments, protein-substrate complexes, protein-protein complexes, biotin-binding proteins, biotin-streptavidin complexes, peptides, ligands, receptors, aptamers, nucleic acid-based aptamers, complementary nucleic acids, carbohydrates, chelates or small molecules.

8. The method according to claim 1, wherein the complementary second agent specifically links to the targeted first agent within the lesion.

9. The method according to claim 8, wherein the second agent has a high affinity binding molecule on its surface, which is specific for the complementary receptor molecule on the first agent.

10. The method according to claim 9, wherein the targeting molecule of the secondary agent can comprise:
ntibodies, antibody fragments, protein-substrate complexes, protein-protein complexes, biotin-binding proteins, biotin-streptavidin complexes, peptides, ligands, receptors, aptamers, nucleic acid-based aptamers, complementary nucleic acids, carbohydrates, chelates or small molecules.

11. The method according to claim 8, wherein the second agent is localized to the first agent by magnetic attraction.

12. The method according to claim 1, wherein the first agent also carries a payload of high Z atoms.

13. The method according to claim 12, wherein the targeted first agent comprises an element having an atomic number (Z) of 39 or greater.

14. The method according to claim 13, wherein the targeting agent comprises Y, In, W, Re, Gd, Sn, La, Ac, Bi Ra, Pm, Sc, Sr, Ra and Pd, Ag, Ir, Pt, Au or a combination thereof.

15. The method according to claim 1, wherein the second agent also carries a payload of high Z atoms.

16. The method according to claim 15, wherein the targeted first agent comprises an element having an atomic number (Z) of 39 or greater.

17. The method according to claim 16, wherein the targeting agent comprises Y, In, W, Re, Gd, Sn, La, Ac, Bi, Ra, Pm, Sc, Sr, Ra and Pd, Ag, Ir, Pt, Au or a combination thereof.

18. The method according to claim 1, wherein the targeting, complementary second agent contains and delivers compounds, which are cytotoxic by themselves.

19. The method according to claim 18, wherein the second agent also carries a payload of cytoxic agent, chemotherapy, immunotherapy, radioisotope or toxin.

20. The method according to claim 1, wherein the targeting, linked second agent contains and delivers compounds, which become cytotoxic upon interaction with radiation.

21. The method according to claim 20, wherein the delivered compounds comprise a radiation dose-enhancing agent.

22. The method according to claim 20, further comprising:
a. measurement of the delivered compounds in the lesion with diagnostic imaging,
b. repeated infusions and measurements of the delivered compounds in the lesion until an optimal threshold concentration of the compounds is reached,
c. determination of radiation dosimetry for treatment using the mass energy-absorption coefficients (MEAC) for the entire radiation spectrum and according to the effects of the tissue type and depth of tissue, as well as the contrast type and depth of contrast, to calculate the dose enhancement factor (DEF),
d. administration of external beam or seed-based radiation therapy to the diseased tissue.

23. The method according to claim 1, wherein a complementary third or subsequent agent specifically targets the second targeting agent within the lesion.

24. The method according to claim 1, wherein the targeting, complementary third agent contains and delivers compounds, which are cytotoxic by themselves.

25. The method according to claim 24, wherein the third agent also carries a payload of cytoxic agent, chemotherapy, immunotherapy, radioisotope or toxin.

26. The method according to claim 1, wherein the targeting, linked third agent contains and delivers compounds, which become cytotoxic upon interaction with radiation.

27. The method according to claim 26, wherein the delivered compounds comprise a radiation dose-enhancing agent.

28. The method according to claim 26, further comprising:
a. measurement of the delivered compounds in the lesion with diagnostic imaging,
b. repeated infusions and measurements of the delivered compounds in the lesion until an optimal threshold concentration of the compounds is reached,
c. determination of radiation dosimetry for treatment using the mass energy-absorption coefficients (MEAC) for the entire radiation spectrum and according to the effects of the tissue type and depth of tissue, as well as the contrast type and depth of contrast, to calculate the dose enhancement factor (DEF),
d. administration of external beam or seed-based radiation therapy to the diseased tissue.

29. The method according to claim 1, for the treatment of primary or metastatic cancers.

30. The method according to claim 1, for the treatment of non-cancerous lesions, vascular plaques, or nervous system lesions.

31. A method according to claim 1, wherein the targeting agent is used to both diagnose and treat pathological lesions in the head or body.

32. The method according to claim 2, wherein the targeting agent is a high payload system selected from the group consisting of a polymer or a particle capable of carrying a high payload of heavy elements and targeting moiety.

33. The method according to claim 32, wherein the polymer is a liposome.

34. The method according to claim 32, wherein the particle is selected from bismuth or gold or platinum or tungsten or iodine or silver or palladium.

35. A system comprising:
a. a targeted first agent, wherein the first agent localizes to diseased tissue; and
b. a complementary second agent, which specifically targets and links to the first agent within the diseased tissue.

36. A system according to claim 35, further comprising:
a. the first agent, wherein the first agent attracts, or enhances, therapeutic concentrations of other linked therapeutic compounds in the diseased tissue relative to the surrounding normal tissues; and
b. a complementary second agent, wherein the second agent contains therapeutic compounds, and also attracts, or enhances, therapeutic concentrations of other linked therapeutic compounds in the diseased tissue relative to the surrounding normal tissues; and
c. a measurement device to measure the delivered compounds in the lesion and determine when an optimal threshold concentration of the compounds is reached.

37. A system according to claim 36, wherein the therapeutic compounds of the complementary second agent are cytotoxic by themselves.

38. A system according to claim 36, wherein the therapeutic compounds of the complementary second agent become cytotoxic upon interaction with radiation.
